(19) United States
(12) Patent Application Publication
(10) Pub. No.: US 2026/0355095 A1
(43) Pub. Date: Nov. 12, 2026

(54) COMPOSITION HAVING EXCELLENT EFFECT OF IMPROVING SKIN ROUGHNESS

(71) Applicant: SHISEIDO COMPANY, LTD., Tokyo (JP)

(72) Inventor: Ayumi ASAI, Tokyo (JP)

(73) Assignee: SHISEIDO COMPANY, LTD., Tokyo (JP)

(21) Appl. No.: 19/133,429

(22) PCT Filed: Dec. 21, 2023
(86) PCT No.: PCT/JP2023/045894

§ 371 Date: May 28, 2025

(30) Foreign Application Priority Data
Dec. 28, 2022 (JP) 2022-212586

(51) Int. Cl. A61K 8/81; A61Q 19/00

(52) U.S. Cl. CPC A61K 8/81; A61Q 19/00; A61K 2800/412; A61K 2800/5426; A61K 2800/594

(57) ABSTRACT

A composition having an excellent effect of improving skin roughness is disclosed. The composition containing a specific polymer exhibiting a pH of a specific range, and having an excellent effect of improving skin roughness.

### REFERENCE TO RELATED APPLICATIONS

The present patent application involves a claim of priority based on Japanese Patent Application No. 2022-212586 (filing date: Dec. 28, 2022), which is a patent application filed earlier in Japan. The entire disclosure in this earlier patent application is incorporated herein by reference.

### TECHNICAL FIELD

The present invention relates to a composition having an excellent effect of improving skin roughness.

### BACKGROUND ART

Environmental changes and diversification of dietary habits cause the serious deterioration of skin conditions such as sensitive skin and atopic dermatitis. There are various possible methods for improving these conditions, one of which is to facilitate improvement by applying to the subject a composition having the effect of improving such skin conditions. As such compositions, various products such as cosmetics having moisturizing effects and skin roughness improving effects have been developed.

For example, PTL 1 discloses a stickiness-reduced external skin preparation characterized by containing at least one type of mucopolysaccharide and a polymer of 2-methacryloyloxyethyl phosphorylcholine. Mucopolysaccharides such as hyaluronic acid and chondroitin sulfate, as well as polyhydric alcohols such as sorbitol, ethylene glycol, and glycerin, organic acid salts such as sodium pyrrolidone carboxylate and sodium lactate, and proteins such as collagen and elastin, are ingredients that have been used as moisturizing agents for the skin.

Additionally, some people may develop dark and/or darker colored spots giving an uneven appearance on their skin, particularly on their hands, at various stages of their lives, such spots are improved by applying to the subject compositions having an effect of improving skin spots, and as such compositions, products of cosmetics having an effect of improving spots have been developed.

For example, PTL 2 discloses cosmetics and/or dermatological composition containing a cosmetically acceptable acidic aqueous medium and at least one crosslinked poly(2-acrylamido-2-methylpropanesulfonic acid) polymer that is at least 90% neutralized. Examples of organic active substances contained in such an acidic aqueous medium include kojic acid, caffeic acid, salicylic acid and derivatives thereof. These ingredients are commonly used in the treatment of spots.

Thus, many studies have been conducted on compositions having the effect of improving skin conditions. However, although the unevenly developed deterioration of skin condition requires relatively extensive care for improvement in the severely deteriorated areas, while relatively less extensive care is sufficient in mildly deteriorated areas, usual care of applying uniformly a topical composition to the areas causes problems such as inability to provide appropriate care depending on the degree of deterioration, and thus there appear many cases in which the effect is not necessarily sufficient.

### CITATION LIST

### Patent Literature

[PTL 1] JP-A-H9-052848
[PTL 2] JP-A-H10-067616

### SUMMARY OF INVENTION

The present inventors have found that a composition having a pH within a specific range and containing a specific polymer has an excellent effect of improving skin roughness. The present invention is based on this finding.

Accordingly, the present invention provides a composition containing a specific polymer exhibiting a pH of a specific range, and having an excellent effect of improving skin roughness.

According to the present invention, the following inventions are provided.
(1) A composition comprising:
   (A) microgel, and
   (B) a copolymer of 2-methacryloyloxyethyl phosphorylcholine and butyl methacrylate,
   wherein the ingredient (A)
(a1) contains a polymer having one or more groups of sulfonate group or sulfate group and
(a2) is amorphous in the composition,
   the composition exhibiting a pH of 4.0 to 5.0.
(2) The composition according to (1), wherein the polymer in (a1) is a copolymer with a monomer of a compound having one or more groups of sulfonate group or sulfate group.
(3) The composition according to (1) or (2), wherein the monomer in (a1) is ammonium acryloyldimethyltaurate.
(4) The composition according to any one of (1) to (3), wherein the copolymer in (a1) is one or more selected from the group consisting of (ammonium acryloyldimethyltaurate/beheneth-25 methacrylate) crosspolymer and (ammonium acryloyldimethyltaurate/vinylpyrrolidone) copolymer.
(5) The composition according to any one of (1) to (4), wherein the ingredient (A) has an average particle size of 2 to 50 μm in a water-swollen state.
(6) The composition according to any one of (1) to (5), wherein an MA/MB ratio in the composition is 1.0 or more, where MA is the amount of ingredient (a1) and MB is the amount of ingredient (B).
(7) The composition according to any one of (1) to (6), further comprising (C) a polyhydric alcohol.
(8) The composition according to (7), wherein the ingredient (C) is one or more selected from the group consisting of glycerin and polyglycene.
(9) The composition according to (7) or (8), wherein the ingredient (C) further contains one or more selected from the group consisting of dihydric alcohols having 3 to 6 carbon atoms.
(10) The composition according to any one of (1) to (9), wherein the composition is in the form of a solution, suspension, dispersion, emulsion, cream, ointment, gel, lotion, foam, paste, or spray.
(11) The composition according to any one of (1) to (10), wherein the composition is for cosmetics.
(12) A composition comprising:
   (A) microgel,
   wherein the ingredient (A)
(a1) contains a polymer having one or more groups of sulfonate group or sulfate group,
(a3) contains a cationic polymer,
(a4) has an average particle size of 2 to 50 μm in a water-swollen state, and
contains the ingredients (a1) and (a3) blended together so that the total charge of the ingredient (A) is negative in the composition,
   the composition exhibiting a pH of 4.0 to 5.0.
(13) The composition according to (12), wherein the polymer in (a1) is a copolymer with a monomer of a compound having one or more groups of sulfonate group and sulfate group.
(14) The composition according to (12) or (13), wherein the monomer in (a1) is sodium acryloyldimethyltaurate.
(15) The composition according to any one of (12) to (14), wherein the copolymer in (a1) is a (sodium acryloyldimethyltaurate/dimethylacrylamide) crosspolymer.
(16) The composition according to any one of (12) to (15), wherein the average particle size of the ingredient (a1) is less than 2 μm in a water-swollen state, as measured by a laser analysis particle size distribution analyzer.
(17) The composition according to any one of (12) to (16), wherein the cationic polymer in (a3) is one or more selected from the group consisting of cationized hydroxyalkyl cellulose, cationized cellulose, cationized guar gum, cationized locust bean gum, cationized chitosan, cationized oligosaccharides, cationized starch derivatives, acrylamide/acrylic acid/dimethyldiallylammonium chloride copolymer, and vinylpyrrolidone/N,N-dimethylaminoethyl methacrylic acid copolymer sulfate.
(18) The composition according to any one of (12) to (17), wherein the ingredient (A) is amorphous in the composition.
(19) The composition according to any one of (12) to (18), further comprising (C) a polyhydric alcohol.
(20) The composition according to (19), wherein the ingredient (C) is one or more selected from the group consisting of glycerin and polyglycene.
(21) The composition according to (19) or (20), wherein the ingredient (C) further contains one or more selected from the group consisting of dihydric alcohols having 3 to 6 carbon atoms.
(22) The composition according to any one of (12) to (21), wherein the composition is in the form of a solution, suspension, dispersion, emulsion, cream, ointment, gel, lotion, foam, paste, or spray.
(23) The composition according to any one of (12) to (22), wherein the composition is for cosmetics.

According to the present invention, there is provided a composition containing a specific polymer exhibiting a pH of a specific range, and having an excellent effect of improving skin roughness. Use of the composition of the present invention enables skin roughness to be effectively improved. In addition, use of the composition of the present invention also enables the skin to be effectively moisturized.

### DESCRIPTION OF EMBODIMENTS

According to one embodiment of the present invention, there is provided a composition comprising (A) microgel and (B) a copolymer of 2-methacryloyloxyethyl phosphorylcholine and butyl methacrylate. Ingredient (A) in the composition includes (a1) a polymer having one or more groups of sulfonate group or sulfate group, and (a2) is amorphous in the composition. In addition, the composition exhibits a pH of 4.0 to 5.0. In the present invention, the term “amorphous” refers to shapes other than those having a regular shape such as spheres (for example, spheres synthesized by emulsion polymerization), fragments, plates, scales, needles, etc.

According to another embodiment of the present invention, there is provided a composition comprising (A) microgel. Ingredient (A) in the composition includes (a1) a polymer having one or more groups of sulfonate group or sulfate group, (a3) a cationic polymer, and (a4) has an average particle size of 2 to 50 μm in a water-swollen state. The composition exhibits a pH of 4.0 to 5.0. The average particle size of the microgel in this composition in a water-swollen state means the particle size at a cumulative percentage of 50% in the particle size distribution, and is a value measured using an aqueous solution containing 0.05 mass % of NaCl as a solvent. The measurement can be performed by a laser analysis type particle size distribution meter, and other known methods (for example, measurement by image analysis of microscope observation, etc.) may also be used. In such measurements, when the microgel has a spherical shape, its diameter (outer diameter) is taken as the particle size, and when the microgel has a shape, including an amorphous shape, other than spherical shape, the longest part of the shape may be taken as the particle size. This average particle size is the average particle size of the (A) microgel containing both the (a1) ingredient and the (a3) ingredient, and those not containing the (a3) ingredient are not used for calculating the average particle size.

The composition of the present invention comprises microgel. Generally, the term “microgel” refers to internally crosslinked polymer particles that have liquid-like fluidity and forms an aggregate of fine granular gel. This microgel maintains the structural characteristics of polymer gels, such as a three-dimensional cross-linked structure and a hardly changed surface while having the characteristic of colloidal dispersion in a solvent.

The polymer having one or more groups of sulfonate group or sulfate group in the present invention, though not limited to, may have a structure in which a sulfonate group or a salt thereof, or a sulfate group or a salt thereof is bound via a spacer to a main chain of a polymer such as polyacrylic acid or polymethacrylic acid, may be a polymer having naturally derived sulfonate/sulfate group, or may be a thickener. The polymer having one or more groups of sulfonate group or sulfate group in the present invention is preferably a thickener having a sulfonate group bonded thereto, and even among the sulfonate group, more preferably a thickener having taurine or a salt thereof bonded thereto from the viewpoints of stability, safety, etc.

The polymer having one or more groups of sulfonate group or sulfate group in the present invention, though not limited to, is preferably a copolymer with a monomer of a compound having one or more groups of sulfonate group or sulfate group. Specific examples of the monomer in such a copolymer, though not limited to, include preferably ammonium acryloyldimethyltaurate. Furthermore, specific examples of such copolymers include, though not limited to, preferably (ammonium acryloyldimethyltaurate/beheneth-25 methacrylate) crosspolymer, (ammonium acryloyldimethyltaurate/vinylpyrrolidone) copolymer, (sodium acryloyldimethyltaurate/dimethylacrylamide) crosspolymer, etc. For these copolymers, though not limited to, commercially available ones may be used.

The shape of the polymer having one or more groups of sulfonate group or sulfate group in the present invention, though not limited to, may be a definite shape such as a sphere, a fragment, a plate, a scale, or a needle, or may be amorphous. The polymer having one or more groups of sulfonate group or sulfate group in the composition of the present invention may also have an average particle size of less than 2 μm in a water-swollen state. The average particle size of the polymer having one or more groups of sulfonate group or sulfate group in a water-swollen state means the particle size at a cumulative percentage of 50% in the particle size distribution, and is a value measured using an aqueous solution containing 0.05 mass % of NaCl as a solvent. The measurement can be performed by a laser analysis type particle size distribution meter, and other known methods (for example, measurement by image analysis of microscope observation, etc.) may also be used. In such measurements, when the polymer has a spherical shape, its diameter (outer diameter) is taken as the particle size, and when the polymer has a shape, including an amorphous shape, other than spherical shape, the longest part of the shape may be taken as the particle size.

For the copolymer of 2-methacryloyloxyethyl phosphorylcholine and butyl methacrylate in the present invention, though not limited to, one designated as Polyquaternium-51 in cosmetic product names may be used. Furthermore, if there are those commercially available, they may be used, and examples thereof include Lipidure PMB (manufactured by NOF Corporation).

Examples of the cationic polymer in the present invention, though not limited to, preferably include one or more selected from the group consisting of cationized hydroxyalkyl cellulose, cationized cellulose, cationized guar gum, cationized locust bean gum, cationized chitosan, cationized oligosaccharides, cationized starch derivatives, acrylamide/acrylic acid/dimethyldiallylammonium chloride copolymer, and vinylpyrrolidone/N,N-dimethylaminoethyl methacrylic acid copolymer sulfate, and more preferably one or more selected from the group consisting of cationized hydroxyalkyl cellulose (e.g., O-[2-hydroxy-3-(trimethylammonio)propyl]hydroxyethyl cellulose chloride, etc.) and vinylpyrrolidone/N,N-dimethylaminoethyl methacrylic acid copolymer sulfate. For these cationic polymers, though not limited to, commercially available ones may be used.

The pH of the composition of the present invention is not limited as long as it is within the range of 4.0 to 5.0.

In the composition of the present invention containing (A) microgel and (B) a copolymer of 2-methacryloyloxyethyl phosphorylcholine and butyl methacrylate, an MA/MB ratio, though not limited to, is preferably 1.0 or more, and more preferably 1.5 to 8, where MA is the blending amount of ingredient (a1) and MB is the blending amount of ingredient (B).

Thus, according to one preferred embodiment of the present invention, there is provided a composition comprising (A) microgel and (B) a copolymer of 2-methacryloyloxyethyl phosphorylcholine and butyl methacrylate. Ingredient (A) in the composition includes (a1) a polymer having one or more groups of sulfonate group or sulfate group, and (a2) is amorphous in the composition. In addition, the composition exhibits a pH of 4.0 to 5.0. In addition, an MA/MB ratio in the composition is 1.0 or more, where MA is the blending amount of ingredient (a1) and MB is the blending amount of ingredient (B).

In the composition of the present invention containing (A) microgel and (B) a copolymer of 2-methacryloyloxyethyl phosphorylcholine and butyl methacrylate, the microgel, though is not limited to, preferably has an average particle size of 2 to 50 μm in a water-swollen state. The average particle size of the microgel in this composition in a water-swollen state means the particle size at a cumulative percentage of 50% in the particle size distribution, and is a value measured using an aqueous solution containing 0.05 mass % of NaCl as a solvent. The measurement can be performed by a laser analysis type particle size distribution meter, and other known methods (for example, measurement by image analysis of microscope observation, etc.) may also be used. In such measurements, when the microgel has a spherical shape, its diameter (outer diameter) is taken as the particle size, and when the microgel has a shape, including an amorphous shape, other than spherical shape, the longest part of the shape may be taken as the particle size.

The composition of the present invention comprises (A) microgel, in which ingredient (A) contains (a1) a polymer having one or more groups of sulfonate group or sulfate group, (a3) a cationic polymer, and (a4) has an average particle size of 2 to 50 μm in a water-swollen state, and, though not limited to, preferably contains ingredients (a1) and (a3) blended together so that the total charge of ingredient (A) in the composition is negative in the composition.

In the present invention, the “total charge is negative” in ingredient (A) in the composition means that the total charge, obtained by adding up the charges of all particles of ingredient (A) contained in the composition, is negative. The method for making the total charge negative is not particularly limited, and may be, for example, by blending ingredients (a1) and (a3) so that ingredient (a1) is contained in a ratio range greater than the blending ratio in which the charge is neutralized and the yield of an aggregate is largest.

In addition, the composition of the present invention comprises (A) microgel, in which ingredient (A) contains (a1) a polymer having one or more groups of sulfonate group or sulfate group, (a3) a cationic polymer, and (a4) has an average particle size of 2 to 50 μm in a water-swollen state, and, though not limited to, ingredient (a1) preferably has an average particle size of less than 2 μm in a water-swollen state.

Therefore, according to another preferred embodiment of the present invention, there is provided a composition comprising (A) microgel. Ingredient (A) in the composition includes (a1) a polymer having one or more groups of sulfonate group or sulfate group, (a3) a cationic polymer, and (a4) has an average particle size of 2 to 50 μm in a water-swollen state. In addition, the composition exhibits a pH of 4.0 to 5.0. In addition, ingredients (a1) and (a3) are mixed together so that the total charge of ingredient (A) in the composition is negative in the composition.

Furthermore, according to another preferred embodiment of the present invention, there is provided a composition comprising (A) microgel. Ingredient (A) in the composition includes (a1) a polymer having one or more groups of sulfonate group or sulfate group, (a3) a cationic polymer, and (a4) has an average particle size of 2 to 50 μm in a water-swollen state. In addition, the composition exhibits a pH of 4.0 to 5.0. In addition, the average particle size of ingredient (a1) is less than 2 μm in a water-swollen state.

Furthermore, according to another preferred embodiment of the present invention, there is provided a composition comprising (A) microgel. Ingredient (A) in the composition includes (a1) a polymer having one or more groups of sulfonate group or sulfate group, (a3) a cationic polymer, and (a4) has an average particle size of 2 to 50 μm in a water-swollen state. In addition, the composition exhibits a pH of 4.0 to 5.0. In addition, ingredients (a1) and (a3) are mixed together so that the total charge of ingredient (A) in the composition is negative in the composition. Furthermore, the average particle size of ingredient (a1) is less than 2 μm in a water-swollen state.

Furthermore, according to another preferred embodiment of the present invention, there is provided a composition comprising (A) microgel. Ingredient (A) in the composition includes (a1) a polymer having one or more groups of sulfonate group or sulfate group, (a3) a cationic polymer, and (a4) has an average particle size of 2 to 50 μm in a water-swollen state. The composition exhibits a pH of 4.0 to 5.0. Furthermore, (A) microgel in the composition is amorphous. In addition, in such a composition, ingredients (a1) and (a3) may be blended so that the total charge of ingredient (A) is negative in the composition, and/or the average particle size of ingredient (a1) may be less than 2 μm in a water-swollen state.

The composition of the present invention may further contain a polyhydric alcohol. Such polyhydric alcohols are not limited as long as they are alcohols having two or more hydroxyl groups in the same molecule, and examples thereof include glycerin, diglycerin, polyglycerin, 1,3-butylene glycol, ethylene glycol, propylene glycol, dipropylene glycol, polyethylene glycol, polypropylene glycol, polyethylene glycol/polypropylene glycol, trimethylolethane, trimethylolpropane, erythritol, pentaerythritol, sorbitan, glucose, sorbitol, maltitol, sucrose, raffinose, hexylene glycol, 1,2-pentanediol, and trehalose. The composition of the present invention preferably contains, as the polyhydric alcohol, one or more selected from the group consisting of glycerin and polyglycene, and more preferably further contains one or more selected from the group consisting of dihydric alcohols having 3 to 6 carbon atoms.

The composition of the present invention may be, though not limited to, a cosmetic composition. The cosmetic composition in the present invention means a composition used for the purpose of make-up.

The compositions of the present invention may be, though not limited to, topical compositions. Used as a topical composition, the composition of the present invention may further contain known ingredients (additives) for blending into topical compositions. Such ingredients include, though not limited to, emulsifiers, hydrating agents, solvents, emollients, stabilizers, thickeners, preservatives, lubricants, chelating agents, fillers, excipients, powders, fragrances, flavors, absorbents, dyes, opacifiers, antioxidants, vitamins, amino acids, neutralizers, buffers, etc.

The method for adjusting the pH of the composition of the present invention may be a general method, though the ingredients are not limited, inorganic and organic alkaline and acidic ingredients used as neutralizers and buffers can be used. Such ingredients may be added in a proper amount to the compositions of the present invention. In the present invention, the proper amount is an amount that is generally used by those skilled in the art when preparing a desired composition, and is not particularly limited.

The topical compositions of the present invention may be liquid, semi-solid or solid, and are preferably in the form of, though not limited to, solution, suspension, dispersion, emulsion, cream, ointment, gel, lotion, foam, paste or spray.

### EXAMPLES

The present invention will be specifically described based on the following examples, but the present invention is not limited to these examples. Unless otherwise specified, the contents are expressed in mass %. The average particle size was measured by a laser analysis particle size distribution analyzer (Microtrac HRA (MicrotracBEL)) and was taken as the cumulative percentage of 50% in the particle size distribution.

### Moisture Retention Evaluation

The test sites were the inner forearms of four healthy subjects in their 40s. Four areas of 2 cm×3 cm were prepared for each subject, and the sites were randomly selected to apply 12 μl of each of the four compositions shown in Table 1. The application site was washed with soap, and the moisture content of the stratum corneum before application of the composition was measured using a Corneometer (Courage+Khazaka). Four hours after application of each composition, the moisture content of the stratum corneum was measured in the same manner. The value before application was taken as 1, and the change after application was calculated.

As shown in Table 1, Comparative Example 2, Comparative Example 3, and Example 1, containing ingredients (A) and/or (B), had higher moisture retention than Comparative Example 1 containing only a solvent. Furthermore, Example 1, containing both ingredient (A) and (B), exhibited a higher moisturizing effect than Comparative Examples 2 and 3, containing ingredient (A) or ingredient (B), respectively.

TABLE 1

| | | | Shape and | Cex | Cex | Cex | Example |
|---|---|---|---|---|---|---|---|
| Ingredient | | Ingredient Name | Properties | 1 | 2 | 3 | 1 |
| 1 | A | (Ammonium | Amorphous | — | 0.1 | — | 0.07 |
| | | acryloyldimethyltaurate/ | micro gel | | | | |
| | | beheneth-25 methacrylate) | | | | | |
| | | crosspolymer (*1) | | | | | |
| | | (average particle size 9.3 μm) | | | | | |
| 2 | B | Polyquaternium-51 solution | Amphoteric | — | — | 5 | 0.6 |
| | | (*2, the pure content of | | | | | |
| | | polyquaternium-51 is 5%) | | | | | |
| 3 | C | Glycerin | | 0.5 | 0.5 | 0.5 | 0.5 |
| 4 | C | BG | | 5 | 5 | 5 | 5 |
| 5 | | Buffer | | Proper | Proper | Proper | Proper |
| | | | | amount | amount | amount | amount |
| 6 | | Water | | Residue | Residue | Residue | Residue |

| | | | | |
|---|---|---|---|---|
| pH | 6.6 | 4.5 | 4.6 | 4.5 |
| Moisture content of stratum corneum after | 1.11 | 1.21 | 1.17 | 1.24 |
| 4 hours (before application = 1) | | | | |
| Significant difference (p < 0.1, same symbols | a | b | b | c |
| indicate no significant difference) | | | | |

*1 Aristoflex HMB (Clariant)
Cex: Comparative example
*2 Lipidure PMB (NOF Corporation)

Evaluation of the Effect of Recovery from Skin Roughness

### Inducing Skin Roughness

The test sites were the inner forearms of healthy individuals in their 20s to 50s. A 2.5 cm×3.4 cm medical cotton pad was soaked in a 10% aqueous solution of sodium lauryl sulfate and applied to the test site, and then covered with a medical moisture-permeable adhesive sheet. One hour after, they were removed, and the test site was washed with water and left open to the air for 2 hours.

### Applying Sample

50 μL of the sample was applied to an area of 3.5 cm×5 cm including the skin roughness-induced area. The sample was applied twice a day, morning and evening, after inducing skin roughness and until the end of the test.

### Evaluation of Recovery Effect

The evaluation was based on the recovery rate of the stratum corneum moisture content. The moisture content of the stratum corneum was measured using a Corneometer (Courage+Khazaka) in the skin roughness-induced area. Measurements were performed before inducing skin roughness, after inducing skin roughness, and 24 or 48 hours after inducing skin roughness and applying the sample, and the recovery rate was evaluated according to the following formula (1).

[Math. 1]

Recovery rate (%)=(Value after applying sample−Value after inducing skin roughness)/(Value before inducing skin roughness−Value after inducing skin roughness)×100 Formula (1)

### Test 1

The constitution of the lotion-like composition sample and the results are shown in Table 2. The test was performed by applying all of the samples to the inside of the forearm of each 10 males, and calculating the average recovery rate 24 hours after. Compared to Reference 1 of a typical lotion-like composition, Examples 2 and 3 satisfying the composition and pH of this invention, exhibited high recovery rates and were considered to have an excellent effect of promoting improvement in the skin roughness-induced areas. In contrast, the high promotion effect as in Examples 2 and 3 was not observed in the following: Comparative Example 4 without ingredient B, Comparative Example 5 using non-microgel without sulfonate group or sulfate group instead of ingredient A, Comparative Example 6 using non-amorphous microgel, and Comparative Example 7 not satisfying the pH conditions in this invention.

TABLE 2

| Ingredient | | Ingredient Name | Remarks | Ref 1 | Ex 2 | Ex 3 | Cex 4 | Cex 5 | Cex 6 | Cex 7 |
|---|---|---|---|---|---|---|---|---|---|---|
| 1 | A | (Ammonium | Amorphous | — | 0.09 | — | 0.12 | — | — | 0.09 |
| | | acryloyldimethyltaurate/ | microgel | | | | | | | |
| | | Beheneth-25 methacrylate) | Average | | | | | | | |
| | | crosspolymer | particle | | | | | | | |
| | | | size: | | | | | | | |
| | | | 8.0 μm | | | | | | | |
| 2 | A | (Ammonium | Amorphous | — | — | 0.07 | — | — | — | — |
| | | acryloyldimethyltaurate/VP) | microgel | | | | | | | |
| | | copolymer | Average | | | | | | | |
| | | | particle | | | | | | | |
| | | | size: | | | | | | | |
| | | | 5.8 μm | | | | | | | |
| 3 | | (Sodium | Spherical | — | — | — | — | — | 0.09 | — |
| | | acrylate/sodium | microgels | | | | | | | |
| | | acryloyldimethyltaurate) | Average | | | | | | | |
| | | copolymer | particle | | | | | | | |
| | | | size: | | | | | | | |
| | | | 3.8 μm | | | | | | | |
| 4 | | Sodium | Non- | — | — | — | — | 0.12 | — | — |
| | | carboxymethylcellulose | microgel | | | | | | | |
| 5 | B | Polyquaternium-51 | Amphoteric | — | 0.03 | 0.05 | — | — | 0.03 | 0.03 |
| | | (5% pure equivalent | | | | | | | | |
| | | content in *2) | | | | | | | | |
| 6 | C | Glycerin | | 0.5 | 0.5 | 0.5 | 0.5 | 0.5 | 0.5 | 0.5 |
| 7 | C | BG | | 5 | 5 | 5 | 5 | 5 | 5 | 5 |
| 8 | | Buffer | | Proper | Proper | Proper | Proper | Proper | Proper | Proper |
| | | | | amount | amount | amount | amount | amount | amount | amount |
| 9 | | Preservative | | Proper | Proper | Proper | Proper | Proper | Proper | Proper |
| | | | | amount | amount | amount | amount | amount | amount | amount |
| 10 | | Chelating agent | | Proper | Proper | Proper | Proper | Proper | Proper | Proper |
| | | | | amount | amount | amount | amount | amount | amount | amount |
| 11 | | Water | | Residue | Residue | Residue | Residue | Residue | Residue | Residue |

| | | | | | | | |
|---|---|---|---|---|---|---|---|
| pH | 6.6 | 4.6 | 4.4 | 4.5 | 6.6 | 4.6 | 6.8 |
| Recovery rate average | 56.3 | 75.7 | 77.5 | 69.5 | 67 | 66.3 | 69.6 |
| Significant difference from | | Exist | Exist | None | None | None | None |
| Ref 1 (p < 0.01) | | | | | | | |

Ref: Reference
Ex: Example
Cex: Comparative example

### Test 2

The constitution of the lotion-like composition sample and the results are shown in Table 3. The test was conducted five times on the same female subjects at different timings. Each sample was applied to different positions of both forearms at each timing, and the average recovery rate 48 hours after was calculated. In each of Examples 4 and 5, the charge neutralization ratio of ingredients (a1) and (a3) was (a1):(a3)=4:6 in terms of ratio by weight of pure ingredients, and the total charge of the ingredients having the ratios shown in Table 3 was negative. Compared to Reference 2 adjusted to a pH of 4.5, Examples 4 and 5 satisfying the composition of this invention, exhibited high recovery rates and were considered to have is an excellent effect of promoting improvement in the skin roughness-induced areas. In contrast, the high improvement effect as in Examples 4 and 5 was not observed in the following: Comparative Example 8 with a polyol but without ingredient A or B, Comparative Examples 9 and 10 with a1 of spherical microgel but without a3, and Comparative Example 11 satisfying the ingredient composition but with pH outside the range of this invention.

TABLE 3

| | | Ingredient | | Ref | Example | Example | Cex | Cex | Cex | Cex |
|---|---|---|---|---|---|---|---|---|---|---|
| Ingredient | | Name | Remarks | 2 | 4 | 5 | 8 | 9 | 10 | 11 |
| 1 | a1 | (Dimethylacrylamide/ | Spherical | — | 0.08 | 0.07 | — | 0.09 | 0.12 | 0.08 |
| | | sodium | microgels | | | | | | | |
| | | acryloyldimethyltaurate) | Average | | | | | | | |
| | | crosspolymer | particle | | | | | | | |
| | | | size: | | | | | | | |
| | | | 1.5 μm | | | | | | | |
| 2 | a3 | Polyquaternium-10 | Cationic | — | 0.04 | — | — | — | — | 0.04 |
| 3 | a3 | Guar | Cationic | — | — | 0.05 | — | — | — | — |
| | | hydroxypropyltrimonium | | | | | | | | |
| | | chloride | | | | | | | | |
| 4 | B | Polyquaternium-51 | Amphoteric | — | — | — | — | 0.03 | — | — |
| | | (5% pure equivalent | | | | | | | | |
| | | content in *2) | | | | | | | | |
| 5 | C | Glycerin | | — | 0.5 | 0.5 | 0.5 | 0.5 | 0.5 | 0.5 |
| 6 | C | BG | | — | 5 | 5 | 5 | 5 | 5 | 5 |
| 7 | | Buffer | | Proper | Proper | Proper | Proper | Proper | Proper | Proper |
| | | | | amount | amount | amount | amount | amount | amount | amount |
| 8 | | Preservative | | Proper | Proper | Proper | Proper | Proper | Proper | Proper |
| | | | | amount | amount | amount | amount | amount | amount | amount |
| 9 | | Chelating agent | | Proper | Proper | Proper | Proper | Proper | Proper | Proper |
| | | | | amount | amount | amount | amount | amount | amount | amount |
| 10 | | Water | | Residue | Residue | Residue | Residue | Residue | Residue | Residue |

| | | | | | | | |
|---|---|---|---|---|---|---|---|
| pH | 4.5 | 4.4 | 4.3 | 4.4 | 4.5 | 4.6 | 6.6 |
| Average particle size of microgels | — | 3.5 μm | 5.1 μm | — | — | — | 3.8 μm |
| formed by a1 and a3 | | | | | | | |
| Recovery rate average | 20.1 | 69.4 | 65.2 | 34.6 | 49.5 | 60.4 | 54.7 |
| Significant difference from | | Exist | Exist | None | None | None | None |
| Ref 2 (p < 0.01) | | | | | | | |

Ref: Reference
Cex: Comparative example

### Other Formulations

### Example 6: Liquid Cosmetics (pH 4.5)

An example of a formulation for liquid cosmetics included in the scope of the composition of the present invention is shown below as Example 6. Ingredients 2 to 8, 12, 13, and 15 to 16 were added to an aliquot of water and the whole was mixed. A mixture of ingredients 9 to 11, 17 and 19 was added thereto and the whole was emulsified using a homogenizer. Thereto was added a solution prepared by dissolving ingredient 1 in the remaining water, adding ingredient 2 and dissolving ingredient 16 thereinto, and then ingredient 18 was added thereto and the whole was mixed uniformly to obtain liquid cosmetics included in the scope of the composition of the present invention. The obtained liquid cosmetics had an excellent effect of recovery from skin roughness.

TABLE 4
Formulation of Example 6

| | | Blending |
|---|---|---|
| No. | Ingredient | Amount |
| 1 | (Ammonium acryloyldimethyltaurate/beheneth-25 | 0.3 |
| | methacrylate) crosspolymer (*1) | |
| 2 | Polyquaternium-51 solution (*2) | 0.1 |
| 3 | Glycerin | 8 |
| 4 | Diglycerin | 1 |
| 5 | BG | 5 |
| 6 | Ethanol | 5 |
| 7 | Highly polymerized polyethylene glycol | 0.2 |
| | (molecular weight approximately 600000) | |
| 8 | (Acrylates/alkyl acrylate (C10-30)) crosspolymer | 0.1 |
| 9 | Dimethicone (about 6cs) | 2 |
| 10 | Triethylhexanoin | 0.5 |
| 11 | Isodecyl neopentanoate | 0.5 |
| 12 | PEG/PPG-17/4 dimethyl ether | 3 |
| 13 | Hydroxyethylimidazolidinone | 1 |
| 14 | Buffer | Proper |
| | | amount |
| 15 | Preservative | Proper |
| | | amount |
| 16 | Chelating agent | Proper |
| | | amount |
| 17 | Neutralizer | Proper |
| | | amount |
| 18 | Stabilizer | Proper |
| | | amount |
| 19 | Flavor | Proper |
| | | amount |
| 20 | Water | Residue |

### Example 7: Lotion (pH 4.7)

An example of a formulation for lotion included in the scope of the composition of the present invention is shown below as Example 7. Ingredient 1 was dissolved in an aliquot of water, and ingredient 2 was added thereto and the whole was mixed. To the remaining water, ingredients 3 to 18 were added and the whole was mixed uniformly, and the previously mixed aqueous solution was added thereto to obtain a lotion included in the scope of the composition of the present invention. The obtained lotion had an excellent effect of recovery from skin roughness.

TABLE 5
Formulation of Example 7

| | | Blending |
|---|---|---|
| No. | Ingredient | Amount |
| 1 | (Ammonium acryloyldimethyltaurate/beheneth-25 | 0.1 |
| | methacrylate) crosspolymer (*1) | |
| 2 | Polyquaternium-51 solution (*2) | 0.03 |
| 3 | Glycerin | 5 |
| 4 | BG | 5 |
| 5 | Dipropylene glycol | 5 |
| 6 | Xanthan gum | 0.1 |
| 7 | Sodium polyacrylate | 0.1 |
| 8 | Erythritol | 1 |
| 9 | Dipotassium glycyrrhizinate | 0.1 |
| 10 | Allantoin | 0.1 |
| 11 | Betaine | 5 |
| 12 | PPG-13 Decyltetradeceth-24 | 0.3 |
| 13 | PEG/PPG-14/7 dimethyl ether | 5 |
| 14 | Buffer | Proper |
| | | amount |
| 15 | Preservative | Proper |
| | | amount |
| 16 | Chelating agent | Proper |
| | | amount |
| 17 | Stabilizer | Proper |
| | | amount |
| 18 | Flavor | Proper |
| | | amount |
| 19 | Water | Residue |

### Example 8: Facial Cleanser (pH 4.7)

An example of a formulation for facial cleanser included in the scope of the composition of the present invention is shown below as Example 8. Ingredient 1 was dissolved in an aliquot of water, and ingredient 2 was added thereto and the whole was mixed. To the remaining water, ingredients 3 to 5, 7, 8, 11, 12 to 15, 17, and 18 were added and the whole was mixed uniformly, and a uniform mixture of ingredients 9, 16, and 19 in ingredient 6 was added thereto and the whole was mixed. Ingredient 10 was added to the mixture and the whole was emulsified using a homogenizer, and the aqueous solutions of ingredients 1 and 2 were added thereto and the whole was mixed to obtain a facial cleanser included in the scope of the composition of the present invention. The resulting facial cleanser had an excellent effect of recovery from skin roughness.

TABLE 6
Formulation of Example 8

| | | Blending |
|---|---|---|
| No. | Ingredient | Amount |
| 1 | (Ammonium acryloyldimethyltaurate/VP) | 1.2 |
| | copolymer (*3) | |
| 2 | Polyquaternium-51 solution (*2) | 0.2 |
| 3 | Glycerin | 12 |
| 4 | BG | 10 |
| 5 | Dipropylene glycol | 8 |
| 6 | Ethanol | 3 |
| 7 | PEG/PPG-14/7 dimethyl ether | 5 |
| 8 | (Acrylates/alkyl acrylate (C10-30)) crosspolymer | 0.08 |
| 9 | PPG-13 decyltetradeceth-24 | 0.7 |
| 10 | Dimethicone (about 20cs) | 0.5 |
| 11 | Sodium methyl cocoyl taurate | 0.2 |
| 12 | Hydrolyzed corn starch | 1 |
| 13 | Xanthan gum | 0.15 |
| 14 | Sodium hyaluronate | 0.05 |
| 15 | Buffer | Proper |
| | | amount |
| 16 | Preservative | Proper |
| | | amount |
| 17 | Chelating agent | Proper |
| | | amount |
| 18 | Stabilizer | Proper |
| | | amount |
| 19 | Flavor | Proper |
| | | amount |
| 20 | Water | Residue |

(*3) Aristoflex AVC (Clariant), average particle size 5.8 μm

### Example 9: Sheet Mask Impregnation Liquid (pH 4.2)

An example of a formulation for a sheet mask impregnation liquid included in the range of the composition of the present invention is shown below as Example 9. Ingredient 1 was dissolved in ingredient 19, ingredient 2 was added thereto and the whole was mixed, and then ingredients 3, 5, and 14 to 18 were added thereto and dissolved. A mixture of ingredient 8 to 13 in ingredient 4 was added thereto and the whole was mixed to obtain a sheet mask impregnation liquid included in the scope of the composition of the present invention. The obtained sheet mask impregnation liquid had an excellent effect of recovery from skin roughness.

TABLE 7
Formulation of Example 9

| | | Blending |
|---|---|---|
| No. | Ingredient | Amount |
| 1 | (Ammonium acryloyldimethyltaurate/VP) | 0.5 |
| | copolymer (*3) | |
| 2 | Polyquaternium-51 solution (*2) | 0.1 |
| 3 | Glycerin | 2 |
| 4 | BG | 9 |
| 5 | PEG-6 | 3 |
| 6 | Methyl gluceth-10 | 0.5 |
| 7 | Sodium methyl cocoyl taurate | 0.1 |
| 8 | Polyglyceryl-2 diisostearate | 0.2 |
| 9 | Isostearic acid | 0.2 |
| 10 | Isostearyl alcohol | 0.2 |
| 11 | PEG-60 hydrogenated castor oil | 0.2 |
| 12 | Hydrogenated polydecene | 0.5 |
| 13 | Lauroyl glutamate di(phytosteryl/octyldodecyl) | 0.1 |
| 14 | Glycylglycine | 1.5 |
| 15 | Buffer | Proper |
| | | amount |
| 16 | Preservative | Proper |
| | | amount |
| 17 | Chelating agent | Proper |
| | | amount |
| 18 | Stabilizer | Proper |
| | | amount |
| 19 | Water | Residue |

### Example 10: Mist-Like Lotion (pH 4.8)

An example of a formulation for mist-like lotion included in the scope of the composition of the present invention is shown below as Example 10. Ingredient 1 was dissolved in ingredient 14, and ingredient 2 was added thereto and the whole was mixed. Ingredients 3 to 6 and 8 to 13 were added thereto and the whole was mixed uniformly, and then ingredient 7 was added thereto and the whole was mixed to obtain a lotion, dischargeable in a mist-like form from a gas-free container using a dispenser, included in the scope of the composition of the present invention. The obtained lotion, dischargeable in a mist-like form from a gas-free container using a dispenser, had an excellent effect of recovery from skin roughness.

TABLE 8
Formulation of Example 10

| | | Blending |
|---|---|---|
| No. | Ingredient | Amount |
| 1 | (Ammonium acryloyldimethyltaurate/VP) | 0.12 |
| | copolymer (*3) | |
| 2 | Polyquaternium-51 solution (*2) | 0.04 |
| 3 | Diglycerin | 1 |
| 4 | BG | 7 |
| 5 | Propanediol | 3 |
| 6 | Ethanol | 5 |
| 7 | (Dimethicone/vinyl dimethicone) crosspolymer | 12 |
| | water dispersion (*4) | |
| 8 | (Acrylates/alkyl acrylate (C10-30)) crosspolymer | 0.06 |
| 9 | PEG-12 dimethicone | 0.4 |
| 10 | Buffer | Proper |
| | | amount |
| 11 | Preservative | Proper |
| | | amount |
| 12 | Chelating agent | Proper |
| | | amount |
| 13 | Neutralizer | Proper |
| | | amount |
| 14 | Water | Residue |

(*4) DOWSIL BY 29-129 (Dow Toray Industries, Inc.)

### Example 11: All-In-One Gel (pH 4.8)

An example of a formulation for all-in-one gel included in the scope of the composition of the present invention is shown below as Example 11. Ingredient 1 was dissolved in an aliquot of water, and ingredient 2 was added thereto and the whole was mixed. An aliquot of water was heated to 90 °C to dissolve ingredient 7, and ingredient 3 was added thereto and the whole was homogenized, and the mixture was then solidified at room temperature and pulverized with a homogenizer to obtain a paste-like gel. The remaining water was heated to 50 °C to dissolve ingredient 9, and after ingredient 5 in which ingredient 8 was dissolved was added thereto, a mixture prepared by heat-dissolving ingredient 6 in ingredient 4 and adding moistened ingredient 17 was added thereto, the whole was mixed uniformly, and a mixture of ingredients 10 to 14 was added thereto and the whole was emulsified using a homogenizer. Ingredients 18 to 20, the previously prepared aqueous solution of ingredients 1 and 2, and the paste-like gel were mixed to obtain an all-in-one gel included in the scope of the composition of the present invention. The obtained all-in-one gel had an excellent effect of recovery from skin roughness.

TABLE 9
Formulation of Example 11

| | | Blending |
|---|---|---|
| No. | Ingredient | Amount |
| 1 | (Ammonium acryloyldimethyltaurate/beheneth-25 | 1 |
| | methacrylate) crosspolymer (*1) | |
| 2 | Polyquaternium-51 solution (*2) | 0.6 |
| 3 | Glycerin | 12 |
| 4 | Dipropylene glycol | 10 |
| 5 | Ethanol | 5 |
| 6 | (PEG-240/Decyltetradeceth-20/HDI) copolymer | 0.7 |
| 7 | Agar | 1 |
| 8 | PEG-12 dimethicone | 1 |
| 9 | Sodium methyl stearoyl taurate | 0.03 |
| 10 | Squalane | 1.5 |
| 11 | Pentaerythrityl tetraethylhexanoate | 1.5 |
| 12 | Dimethicone (about 6cs) | 1.5 |
| 13 | Diphenylsiloxyphenyl trimethicone | 1.5 |
| 14 | Glyceryl diisostearate | 1.5 |
| 15 | 2-O-ethyl ascorbic acid | 0.5 |
| 16 | Serine | 0.3 |
| 17 | Lauroyl lysine | 2 |
| 18 | Buffer | Proper |
| | | amount |
| 19 | Preservative | Proper |
| | | amount |
| 20 | Chelating agent | Proper |
| | | amount |
| 21 | Water | Residue |

### Example 12: Liquid Cosmetics (pH 4.8)

An example of a formulation for liquid cosmetics included in the scope of the composition of the present invention is shown below as Example 12. Ingredient 1 was dissolved in an aliquot of water, and ingredients 2 and 3 were dissolved in another aliquot of water, and then the two aqueous solutions were mixed. The resulting composition had an average particle size of 6.2 μm and a negative total charge. Ingredients 4 to 8 and ingredients 12 to 16 were added to the remaining water and the whole was mixed uniformly, and a mixture of ingredients 9 to 11 and 19 was added thereto, then ingredient 17 was added thereto, and the resulting mixture was emulsified using a homogenizer. The previously prepared aqueous solutions of ingredients 1 to 3 were then added thereto and the whole was mixed to obtain liquid cosmetics included in the scope of the composition of the present invention. The obtained liquid cosmetics had an excellent effect of recovery from skin roughness.

TABLE 10
Formulation of Example 12

| | | Blending |
|---|---|---|
| No. | Ingredient | Amount |
| 1 | (Dimethylacrylamide/sodium acryloyldimethyltaurate) | 0.3 |
| | crosspolymer | |
| 2 | Carrageenan | 0.1 |
| 3 | Polyquaternium-10 | 0.2 |
| 4 | Glycerin | 1 |
| 5 | BG | 5 |
| 6 | Dipropylene glycol | 5 |
| 7 | Erythritol | 1 |
| 8 | (Acrylates/alkyl acrylate (C10-30)) crosspolymer | 0.15 |
| 9 | Dimethicone (about 100cs) | 1 |
| 10 | Triethylhexanoin | 0.5 |
| 11 | Meadowfoam oil | 0.5 |
| 12 | PEG/PPG-14/7 dimethyl ether | 1 |
| 13 | Potassium methoxysalicylate | 1 |
| 14 | Buffer | Proper |
| | | amount |
| 15 | Preservative | Proper |
| | | amount |
| 16 | Chelating agent | Proper |
| | | amount |
| 17 | Neutralizer | Proper |
| | | amount |
| 18 | Stabilizer | Proper |
| | | amount |
| 19 | Flavor | Proper |
| | | amount |
| 20 | Water | Residue |

### Example 13: Lotion (pH 4.5)

An example of a formulation for a lotion included in the scope of the composition of the present invention is shown below as Example 13. The resulting composition had an average particle size of 2.2 μm and a negative total charge. Ingredient 2 was dissolved in an aliquot of water, ingredient 1 was dissolved in the remaining water, and both were mixed with each other. Ingredient 17 was added and dissolved in the mixture, thereto added and uniformly mixed were moistened ingredients 6 and 7 added to a mixture of ingredients 4 and 16; a mixture prepared by dissolving ingredients 8 to 10 in heated ingredient 5, and adding ingredients 13, 14 and 19; and ingredient 3; and then ingredient 18 was dissolved thereto to obtain a lotion within the scope of the composition of the present invention. The obtained lotion had an excellent effect of recovery from skin roughness.

TABLE 11
Formulation of Example 13

| | | Blending |
|---|---|---|
| No. | Ingredient | Amount |
| 1 | (Dimethylacrylamide/sodium acryloyldimethyltaurate) | 0.1 |
| | crosspolymer | |
| 2 | Polyquaternium-10 | 0.05 |
| 3 | Glycerin | 5 |
| 4 | BG | 5 |
| 5 | Dipropylene glycol | 5 |
| 6 | Xanthan gum | 0.1 |
| 7 | Tremella fuciformis polysaccharide | 0.05 |
| 8 | PPG-8 Cetes-20 | 0.1 |
| 9 | PEG-12 dimethicone | 0.05 |
| 10 | PEG-10 phytosterol | 0.02 |
| 11 | PEG/PPG-17/4 dimethyl ether | 2 |
| 12 | Tranexamic acid | 1 |
| 13 | Tocopherol acetate | 0.1 |
| 14 | Panthenyl ethyl | 0.05 |
| 15 | Buffer | Proper |
| | | amount |
| 16 | Preservative | Proper |
| | | amount |
| 17 | Chelating agent | Proper |
| | | amount |
| 18 | Stabilizer | Proper |
| | | amount |
| 19 | Flavor | Proper |
| | | amount |
| 20 | Water | Residue |

### Example 14: Pack (pH 4.9)

An example of a formulation for a pack included in the scope of the composition of the present invention is shown below as Example 14. Ingredient 1 was dissolved in an aliquot of water, ingredient 2 was dissolved in another aliquot of water, and both were mixed with each other. The resulting composition had an average particle size of 4.0 μm and a negative total charge. To the remaining water, added and mixed were ingredient 3; ingredient 8 heat dissolved in ingredient 4; ingredients 5 to 7 and ingredients 11 to 14; and then ingredients 9, 10, and 20 were added thereto and the whole was mixed using a homogenizer. Further, ingredients 15 to 19 were added thereto, and then the previously prepared aqueous solutions of ingredients 1 and 2 were added thereto and the whole was mixed to obtain a pack included in the scope of the composition of the present invention. The obtained pack had an excellent effect of recovery from skin roughness.

TABLE 12
Formulation of Example 14

| | | Blending |
|---|---|---|
| No. | Ingredient | Amount |
| 1 | (Dimethylacrylamide/sodium acryloyldimethyltaurate) | 0.5 |
| | crosspolymer | |
| 2 | Polyquaternium-10 | 0.3 |
| 3 | Glycerin | 1 |
| 4 | Dipropylene glycol | 5 |
| 5 | Ethanol | 7 |
| 6 | PEG/PPG-14/7 dimethyl ether | 5 |
| 7 | (Acrylates/alkyl acrylate (C10-30)) crosspolymer | 0.1 |
| 8 | Hydrogenated lecithin | 0.2 |
| 9 | Cetyl ethylhexanoate | 0.5 |
| 10 | Dimethicone (about 6cs) | 0.7 |
| 11 | Hydroxypropyl methylcellulose | 0.2 |
| 12 | Hydrolyzed corn starch | 2 |
| 13 | Carbomer | 0.5 |
| 14 | Cellulose powder | 10 |
| 15 | Buffer | Proper |
| | | amount |
| 16 | Preservative | Proper |
| | | amount |
| 17 | Chelating agent | Proper |
| | | amount |
| 18 | Neutralizer | Proper |
| | | amount |
| 19 | Stabilizer | Proper |
| | | amount |
| 20 | Flavor | Proper |
| | | amount |
| 21 | Water | Residue |

### Example 15: Whole Body Lotion (pH 4.2)

An example of a formulation for a whole body lotion included in the scope of the composition of the present invention is shown below as Example 15. Ingredients 2 and 3 were dissolved in an aliquot of water, ingredient 1 was dissolved in the remaining water, and both were mixed with each other. The resulting composition had an average particle size of 20 μm and a negative total charge. Ingredients 4, 6, and 12 to 18 were added thereto, and further added was a mixture prepared by heat dissolving ingredients 10 and 11 in ingredient 5 and then adding ingredients 7 to 9 thereto and 19 and mixing the whole uniformly, to obtain a whole body lotion included in the scope of the composition of the present invention. The obtained whole body lotion had an excellent effect of recovery from skin roughness.

TABLE 13
Formulation of Example 15

| | | Blending |
|---|---|---|
| No. | Ingredient | amount |
| 1 | (Dimethylacrylamide/sodium acryloyldimethyltaurate) | 0.2 |
| | crosspolymer | |
| 2 | Polyquaternium-10 | 0.1 |
| 3 | Starch hydroxypropyltrimonium chloride | 0.02 |
| 4 | Glycerin | 5 |
| 5 | BG | 10 |
| 6 | Erythritol | 0.1 |
| 7 | Hydrogenated polydecene | 0.3 |
| 8 | Isostearic acid | 0.4 |
| 9 | Retinol acetate | 0.2 |
| 10 | PEG-30 phytosterol | 0.2 |
| 11 | Sorbitan sesquiisostearate | 0.03 |
| 12 | Niacinamide | 1 |
| 13 | Bis-ethoxydiglycol cyclohexane 1,4-dicarboxylate | 1 |
| 14 | Rosa canina fruit extract | 0.5 |
| 15 | Buffer | Proper |
| | | amount |
| 16 | Preservative | Proper |
| | | amount |
| 17 | Chelating agent | Proper |
| | | amount |
| 18 | Stabilizer | Proper |
| | | amount |
| 19 | Flavor | Proper |
| | | amount |
| 20 | Water | Residue |

### Example 16: Emulsion (pH 4.1)

An example of a formulation for emulsion included in the scope of the composition of the present invention is shown below as Example 16. Ingredients 2 and 3 were dissolved in an aliquot of water, ingredient 1 was dissolved in another aliquot of water, and both were mixed with each other. The resulting composition had an average particle size of 5.1 μm and a negative total charge. The remaining water was heated to 70 °C, and a dispersion of ingredient 16 in ingredient 5 was added thereto and dissolved, and then ingredients 15, 18, 20, and 21 were dissolved therein. Thereto were added a mixture formed by heating to 70 °C and dissolving ingredients 7 to 14, then a mixture formed by dissolving ingredients 4 and 17 and ingredients 6 and 19, the whole was emulsified using a homogenizer, the previously prepared aqueous solution of ingredients 1 to 3 was added thereto, and the resulting mixture was cooled to 35 °C to obtain an emulsion included in the scope of the composition of the present invention. The obtained emulsion had an excellent effect of recovery from skin roughness.

TABLE 14
Formulation of Example 16

| | | Blending |
|---|---|---|
| No. | Ingredient | Amount |
| 1 | (Dimethylacrylamide/sodium acryloyldimethyltaurate) | 0.12 |
| | crosspolymer | |
| 2 | Polyquaternium-10 | 0.03 |
| 3 | Polyquaternium-39 solution (10% pure equivalent | 0.1 |
| | content) *5 | |
| 4 | Glycerin | 3 |
| 5 | BG | 7 |
| 6 | Ethanol | 5 |
| 7 | Behenyl alcohol | 1 |
| 8 | Stearyl alcohol | 0.5 |
| 9 | Isododecane | 2 |
| 10 | Pentaerythrityl tetraethylhexanoate | 2 |
| 11 | Dimethicone (about 6cs) | 3 |
| 12 | Diphenylsiloxyphenyl trimethicone | 2 |
| 13 | Glyceryl stearate | 0.5 |
| 14 | Polysorbate 60 | 0.6 |
| 15 | Sodium methyl stearoyl taurate | 0.2 |
| 16 | Succinoglucan | 0.1 |
| 17 | 3-O-ethyl ascorbic acid | 1 |
| 18 | Buffer | Proper |
| | | amount |
| 19 | Preservative | Proper |
| | | amount |
| 20 | Chelating agent | Proper |
| | | amount |
| 21 | Water | Residue |

*5 Merquat ™ 3330PR Polymer (Lubrizol)

1. A composition comprising:
(A) microgel, and
(B) a copolymer of 2-methacryloyloxyethyl phosphorylcholine and butyl methacrylate,
wherein the ingredient (A)
(a1) contains a polymer having one or more groups of sulfonate group or sulfate group and
(a2) is amorphous in the composition,
the composition exhibiting a pH of 4.0 to 5.0.

2. The composition according to claim 1, wherein the polymer in (a1) is a copolymer with a monomer of a compound having one or more groups of sulfonate group or sulfate group.

3. The composition according to claim 2, wherein the monomer in (a1) is ammonium acryloyldimethyltaurate.

4. The composition according to claim 2, wherein the copolymer in (a1) is one or more selected from the group consisting of (ammonium acryloyldimethyltaurate/beheneth-25 methacrylate) crosspolymer and (ammonium acryloyldimethyltaurate/vinylpyrrolidone) copolymer.

5. The composition according to claim 1, wherein the ingredient (A) has an average particle size of 2 to 50 m in a water-swollen state.

6. The composition according to claim 1, wherein an MA/MB ratio in the composition is 1.0 or more, where MA is the amount of ingredient (a1) and MB is the amount of ingredient (B).

7. The composition according to claim 1, further comprising (C) a polyhydric alcohol.

8. The composition according to claim 7, wherein the ingredient (C) is one or more selected from the group consisting of glycerin and polyglycene.

9. The composition according to claim 8, wherein the ingredient (C) further contains one or more selected from the group consisting of dihydric alcohols having 3 to 6 carbon atoms.

10. The composition according to claim 1, wherein the composition is in the form of a solution, suspension, dispersion, emulsion, cream, ointment, gel, lotion, foam, paste, or spray.

11. The composition according to claim 1, wherein the composition is for cosmetics.

12. A composition comprising:
(A) microgel,
wherein the ingredient (A)
(a1) contains a polymer having one or more groups of sulfonate group or sulfate group,
(a3) contains a cationic polymer,
(a4) has an average particle size of 2 to 50 m in a water-swollen state, and
contains the ingredients (a1) and (a3) blended together so that the total charge of the ingredient (A) is negative in the composition,
the composition exhibiting a pH of 4.0 to 5.0.

13. The composition according to claim 12, wherein the polymer in (a1) is a copolymer with a monomer of a compound having one or more groups of sulfonate group or sulfate group.

14. The composition according to claim 13, wherein the monomer in (a1) is sodium acryloyldimethyltaurate.

15. The composition according to claim 13, wherein the copolymer in (a1) is a (sodium acryloyldimethyltaurate/dimethylacrylamide) crosspolymer.

16. The composition according to claim 12, wherein the average particle size of the ingredient (a1) is less than 2 μm in a water-swollen state, as measured by a laser analysis particle size distribution analyzer.

17. The composition according to claim 12, wherein the cationic polymer in (a3) is one or more selected from the group consisting of cationized hydroxyalkyl cellulose, cationized cellulose, cationized guar gum, cationized locust bean gum, cationized chitosan, cationized oligosaccharides, cationized starch derivatives, acrylamide/acrylic acid/dimethyldiallylammonium chloride copolymer, and vinylpyrrolidone/N,N-dimethylaminoethyl methacrylic acid copolymer sulfate.

18. The composition according to claim 12, wherein the ingredient (A) is amorphous in the composition.

19. The composition according to claim 12, further comprising (C) a polyhydric alcohol.

20. The composition according to claim 19, wherein the ingredient (C) is one or more selected from the group consisting of glycerin and polyglycene.

21. The composition according to claim 20, wherein the ingredient (C) further contains one or more selected from the group consisting of dihydric alcohols having 3 to 6 carbon atoms.

22. The composition according to claim 12, wherein the composition is in the form of a solution, suspension, dispersion, emulsion, cream, ointment, gel, lotion, foam, paste, or spray.

23. The composition according to claim 12, wherein the composition is for cosmetics.
